(19) United States
(12) Patent Application Publication
(10) Pub. No.: US 2026/0146967 A1
(43) Pub. Date: May 28, 2026

(54) DEVICE AND SENSOR FOR DETECTION OF AN ANALYTE

(71) Applicant: REMINDED, INC., San Francisco, CA (US)

(72) Inventors: Sanjida YEASMIN, Jersey City, NJ (US); Syed Adil SHAH, Espoo (FI); Annika LUNDSTRÖM, San Francisco, CA (US)

(73) Assignee: REMINDED, INC., San Francisco, CA (US)

(21) Appl. No.: 19/442,610

(22) Filed: Jan. 7, 2026

Related U.S. Application Data
(63) Continuation of application No. PCT/US2025/045324, filed on Sep. 8, 2025.
(60) Provisional application No. 63/691,339, filed on Sep. 6, 2024.

(51) Int. Cl. G01N 27/327; G01N 33/543

(52) U.S. Cl. CPC G01N 27/3276; G01N 27/3277; G01N 27/3278; G01N 33/5438

(57) ABSTRACT

A sensor for detecting an analyte in a sample is provided. The sensor includes a substrate and an electrochemical cell disposed on the substrate. The electrochemical cell includes a working electrode having a recognition layer that can bind to the analyte. The binding of the analyte to the recognition layer can include binding of the analyte to a nuclear receptor immobilized on the recognition layer to induce a conformation change or binding to a binding cavity complementary to the analyte. The sensor further includes a measurement circuit operably coupled to the electrochemical cell that applies a potential to the working electrode; and detects a change in an electrical property at the working electrode caused by the binding of the analyte and the binding correlates to a concentration of the analyte in the sample. Methods and systems utilizing the sensor are also provided.

### CROSS-REFERENCE TO RELATED APPLICATIONS

This application is a Continuation of International Application No. PCT/US2025/045324, filed on Sep. 8, 2025, which claims priority of U.S. Provisional Patent Application No. 63/691,339, filed on Sep. 6, 2024, all of which are incorporated herein by reference in their entirety.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to electrochemical sensors for detecting an analyte of interest, such as biosensors and medical diagnostics. More particularly, it concerns devices and methods for detecting biomarkers using a hybrid recognition system that integrates full-length biological nuclear receptors (NRs) with synthetic catalyst-embedded molecularly imprinted polymers (MIPs) to improve detection accuracy, robustness, and stability.

### BACKGROUND OF THE INVENTION

Accurate and timely biomarker detection is critical for disease diagnosis and health monitoring. Current laboratory-based methods, including Liquid Chromatography-Mass Spectrometry (LC-MS) and Enzyme-Linked Immunosorbent Assay (ELISA), while accurate, are unsuitable for point-of-care applications. These techniques are time-intensive, require sophisticated equipment and trained personnel, and rely on centralized infrastructure, resulting in delayed results that preclude real-time and continuous monitoring.

A further limitation of such laboratory methods is that they provide only single-point measurements. For many conditions, including metabolic disorders, inflammatory diseases, and chronic stress, biomarker levels fluctuate dynamically, requiring frequent or continuous monitoring. Existing techniques do not adequately address this clinical need.

Electrochemical biosensors offer promise for decentralized, portable diagnostics. However, most commercial biosensors employ antibodies as recognition elements. Antibody-based systems present significant disadvantages: (i) they do not undergo intrinsic conformational changes upon binding, necessitating labeling strategies for signal generation; (ii) their large molecular size and random surface orientation hinder electron transfer; (iii) they exhibit batch-to-batch variability and limited thermal stability, resulting in reduced reproducibility.

Molecularly imprinted polymers (MIPs) have been developed as synthetic alternatives due to their low cost, high stability, and chemical robustness. However, conventional MIP-based sensors also face challenges: (i) many rely on external redox mediators for signal generation, increasing assay complexity; (ii) heterogeneous binding sites result in variable affinities; and (iii) cross-reactivity with structurally similar molecules can compromise specificity in complex biofluids such as saliva, sweat, or blood.

Nuclear receptors (NRs) represent a unique class of biological receptors that undergo significant conformational reorganization upon ligand binding. This intrinsic property enables direct, label-free electrochemical transduction.

There is a need for a biosensing platform that combines the high specificity and conformational signal transduction of nuclear receptors with the durability and processability of molecularly imprinted polymers. The present invention addresses this need by providing a hybrid biosensor architecture that offers internal signal verification, expanded dynamic range, and improved stability in real-world conditions.

Citation or identification of any document in this application is not an admission that such document is available as prior art to the present invention.

### SUMMARY OF THE INVENTION

In one aspect of the present invention, a sensor for detection of an analyte in a sample is provided. In some embodiments, the sensor comprises:
a substrate;
an electrochemical cell disposed on the substrate, wherein the electrochemical cell comprises:
   a working electrode comprising:
      a recognition layer disposed on a surface of the working electrode capable of binding to the analyte;
   a reference electrode; and
   a counter electrode;
wherein binding of the analyte to the recognition layer comprises:
binding of the analyte to a nuclear receptor immobilized on the recognition layer thereby inducing a conformation change; or
binding of the analyte to a binding cavity complementary to the analyte; and
a measurement circuit operably coupled to the electrochemical cell wherein the measurement circuit is configured to:
   i) apply a potential to the working electrode; and
   ii) detect a change in an electrical property at the working electrode caused by the binding of the analyte to the recognition layer, wherein said binding is correlative to a concentration of the analyte in the sample.

In some embodiments, the sensor comprises the nuclear receptor, wherein the nuclear receptor is selected from a glucocorticoid receptor (GR), estrogen receptor (ER), progesterone receptor (PR), androgen receptor (AR), mineralocorticoid receptor (MR), thyroid hormone receptor (TR), a peroxisome proliferator-activated receptor (PPAR), a liver X receptor (LXR), a farnesoid X receptor (FXR), a pregnane X receptor (PXR), or a vitamin D receptor (VDR).

In some embodiments, the analyte is selected from cortisol, estradiol, progesterone, testosterone, thyroid hormone, cholesterol, xenobiotics, or vitamin D.

In some embodiments, the sample is selected from saliva, sweat, urine, blood, serum, plasma, or interstitial fluid.

In some embodiments, the measurement circuit is configured to detect the change in the electrical property via electrochemical impedance spectroscopy (EIS), capacitance spectroscopy, field-effect transduction, cyclic voltammetry (CV), differential pulse voltammetry (DPV), square wave voltammetry (SWV), or amperometry.

In some embodiments, the sensor comprises a plurality of working electrodes, wherein each of the plurality of working electrodes is functionalized with a receptor;
wherein each receptor is different from each other; and
wherein each receptor binds to a different analyte than the analyte of the other receptors.

In some embodiments, the receptor of each of the plurality of working electrodes comprises the nuclear receptor.

In some embodiments of the sensor, the analyte comprises cortisol, the nuclear receptor comprises the GR, and the GR is immobilized to the recognition layer. In some embodiments, the GR is immobilized to the recognition layer via physical adsorption.

In some embodiments, the recognition layer comprises a self-assembled monolayer (SAM), wherein the SAM is functionalized for the covalent bonding of the GR to the SAM.

In some embodiments, the SAM comprises a terminal reactive group selected from hydroxyl, carboxyl, amine, or thiol, and wherein the GR is covalently bonded to said terminal reactive group, either directly or through a bifunctional crosslinking agent.

In some embodiments, the SAM comprises mercaptoundecanol (MUDOL), 11-mercaptoundecanoic acid (MUDA), or combinations thereof.

In some embodiments, the sensor comprises MUDA and MUDOL at a molar ratio of MUDA to MUDOL from about 1:1 to 2:1.

In some embodiments, the sensor is a biosensor that is integrated into a wearable device selected from a transdermal patch, a wristband, a tooth-mounted sensor, or a smart textile.

In some embodiments, the sensor further comprises a wireless communication module configured to transmit data corresponding to the analyte concentration to a remote computing device.

In some embodiments, the recognition layer comprises a molecularly imprinted polymer (MIP) layer or a layer of nanoparticles disposed on the working electrode, the MIP comprising a binding cavity complementary to the analyte.

In some embodiments, the MIP further comprises a redox-active catalyst selected from platinum, gold, a metal oxide, a metal-organic framework, graphene, carbon nanotubes, carbon dots, metal nanoparticles, single metal atoms, porphyrins, phthalocyanines, or combinations thereof.

In some embodiments, the catalyst interacts with the analyte captured in the binding cavity to generate a selective electrochemical signal without requiring an external redox mediator.

In some embodiments, the MIP is fabricated by a method selected from emulsion polymerization, precipitation polymerization, sol-gel polymerization, or electrodeposition.

In some embodiments, the sensor is configured to operate in a dual-receptor mode in which signals from both the nuclear receptor and the MIP are measured independently or synergistically.

In some embodiments, the analyte is selected from cortisol, estradiol, progesterone, testosterone, thyroid hormone, vitamin D, dehydroepiandrosterone (DHEA), dopamine, serotonin, norepinephrine, epinephrine, uric acid, lactic acid, tryptophan, kynurenine, kynurenic acid, glutamate, gamma-aminobutyric acid (GABA), brain-derived neurotrophic factor (BDNF), C-reactive protein, IL-6, neuropeptide Y, oxytocin, melatonin, histamine, anandamide, α-amylase, cardiac troponin T, orexin A, or TNF-α.

In another aspect of the present invention, a method for detecting an analyte in a sample is provided. In some embodiments, the method comprising the steps of:
contacting the sample with a sensor comprising an electrochemical cell;
the electrochemical cell comprising a working electrode, the working electrode comprising a nuclear receptor or a molecularly imprinted polymer (MIP) comprising a binding cavity that is complementary to the analyte;
binding the analyte to the nuclear receptor or to the binding cavity of the MIP;
applying a potential to the working electrode;
measuring, via a measurement circuit that is operably coupled to the electrochemical cell, a change in an electrical property at the working electrode, wherein the change in the electrical property is caused by a conformational change of the nuclear receptor induced by binding to the analyte or by catalytic redox activity induced by binding of the analyte to the binding cavity of the MIP; and
correlating the measured change to a concentration of the analyte in the sample.

In some embodiments, the recognition layer comprises the MIP, wherein the MIP comprises an integrated catalyst, and binding of the analyte to the binding cavity promotes selective oxidation or reduction of the analyte without requiring an external redox mediator.

According to another aspect of the present invention, a competitive binding sensor for detecting an analyte in a liquid sample is provided. In some embodiments of the competitive binding sensor, the sensor comprises: a working electrode functionalized with a captured version of the analyte or an analog thereof; a reservoir containing a known quantity of a nuclear receptor protein; a fluidic network configured to mix the liquid sample with the nuclear receptor to form a mixture and deliver the mixture to the working electrode; and
a measurement circuit configured to:
   (i) apply a potential to the working electrode, and
   (ii) measure an electrochemical signal,
wherein the magnitude of the electrochemical signal is inversely proportional to a concentration of the analyte in the liquid sample due to competitive binding between the analyte in the sample and the captured analyte for the nuclear receptor.

In another aspect of the present invention, a multiplexed sensing system is provided. In some embodiments, the multiplexed sensing system comprises: a cartridge interface for receiving a biological sample; a fluidic network configured to distribute the sample to a plurality of electrochemical cells; wherein each electrochemical cell comprises a working electrode functionalized with a different recognition element specific to a different analyte; and a potentiostat unit configured to independently apply a potential to and measure an electrochemical signal from each of the plurality of working electrodes to simultaneously determine concentrations of the plurality of analytes.

In some embodiments of the multiplexed sensing system, the system comprises at least one recognition element comprises a full-length nuclear receptor.

In some embodiments of the multiplexed sensing system, the system at least one recognition element comprises a molecularly imprinted polymer with an integrated catalyst.

In some embodiments, the full-length nuclear receptor is a glucocorticoid receptor (GR) and the analyte comprises cortisol.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112(a)) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 shows a schematic representation of a screen-printed electrode (SPE) configuration suitable for use in the biosensing platform.

FIGS. 2A-2C illustrate various fabrication methods and immobilization strategies for functionalizing working electrodes with biological nuclear receptors (NRs). More in particular, FIG. 2A shows the physical adsorption of the nuclear receptor onto the substrate of the working electrode. FIG. 2B shows the formation of a functionalized SAM that is anchored to the substrate of the working electrode wherein the nuclear receptor is covalently bonded to the SAM. FIG. 2C depicts the nuclear receptor entrapped within a polymer matrix that is subsequently deposited onto the substrate of the working electrode.

FIGS. 3A, 3B, 3C and 3D depict the stepwise fabrication process for creating electrodeposited molecularly imprinted polymer (MIP) films on electrode surfaces.

FIGS. 4A and 4B show two distinct configurations of molecularly imprinted polymers. FIG. 4A depicts electrodeposited MIP films containing a binding cavity that is complementary to the analyte and where the MIP further contains a redox-active catalyst to catalyze a reaction between the analyte and catalyst and generate a measurable signal. FIG. 4B depicts MIP particles doped with a redox active catalyst having a binding cavity specific for a particular analyte, wherein the MIP particles are immobilized to the substrate of the electrode via covalent linkers.

FIG. 5 illustrates the sensing mechanism and signal transduction pathway for nuclear receptor-based (NR) detection, demonstrating the conformational change upon target binding.

FIG. 6 illustrates the sensing mechanism for MIP-based detection, showing the catalytic oxidation/reduction process facilitated by embedded catalyst molecules.

FIG. 7 demonstrates the multiplexing capability of the platform, showing simultaneous detection of multiple biomarkers using an array of differently functionalized working electrodes.

### DETAILED DESCRIPTION OF THE INVENTION

The following detailed description, in conjunction with the appended drawings, sets forth exemplary embodiments of the present invention. These embodiments are provided to convey the principles of the invention to those skilled in the art, and it is to be understood that the invention is not limited to the specific details described herein.

In one aspect, a sensor for detection of an analyte in a sample is provided, wherein the sensor comprises:
a substrate;
an electrochemical cell disposed on the substrate, wherein the electrochemical cell comprises:
   a working electrode comprising:
      a recognition layer disposed on a surface of the working electrode capable of binding to the analyte;
   a reference electrode; and
   a counter electrode;
   wherein binding of the analyte to the recognition layer comprises:
      binding of the analyte to a nuclear receptor immobilized on the recognition layer thereby inducing a conformation change; or
      binding of the analyte to a binding cavity complementary to the analyte; and
a measurement circuit operably coupled to the electrochemical cell wherein the measurement circuit is configured to:
   i) apply a potential to the working electrode; and
   ii) detect a change in an electrical property at the working electrode caused by the binding of the analyte to the recognition layer, wherein said binding is correlative to a concentration of the analyte in the sample.

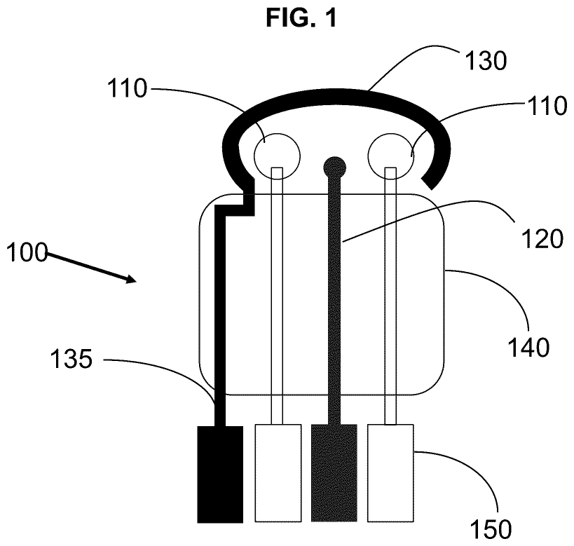

FIG. 1 provides a schematic representation of a sensor 100 according to the present invention. The layout of the sensor typically comprises a working electrode 110, a reference electrode 120, and a counter electrode 130. Each of the working, reference, and counter electrodes are connected via conductive leads 135 to connection pads for interfacing with external measurement electronics, such as a potentiostat. A nonconductive substrate 140 electrically isolates the electrode area from the connection pads. The working electrodes 110 are functionalized with recognition elements (e.g., nuclear receptors or MIPs) where specific binding to a target biomarker occurs. The reference electrode 120 provides a stable potential benchmark, and the counter electrode 130 completes the electrochemical circuit. The sensor platform, in the form of a screen-printed electrode is advantageous for its small size, low cost, mass production, scalability, and disposability.

The analyte that is detected can be an element or a compound. Examples of elements that can be detected include, but are not limited to metals (e.g., lead, polonium), metalloids (e.g., arsenic), and gases (e.g., chlorine). As described further herein, the sample containing an elemental analyte can be in any form such as an aqueous or organic solution containing the analyte (e.g., an aqueous solution containing Pb²⁺). The elemental analyte can be detected via the binding of the analyte to an appropriate receptor to generate a detectable and measurable signal that can be measured by the sensor as described herein.

In some embodiments, the analyte is a compound. Any compound may be detected as long as the analyte is able to bind to a receptor that has a binding affinity for the compound or that is complementary to the analyte (e.g., a binding cavity complementary in shape to the analyte as described herein).

The compound can be any type of compound and can include, for example, organic compounds and inorganic compounds. Organic compounds can include, but are not limited to hydrocarbons, polymers (e.g., PFAS), organometallic compounds, and biomolecules. In some embodiments, the analyte is a biomolecule. In some embodiments, the biomolecule can include, but is not limited to carbohydrates, proteins, nucleic acids, lipids, hormones, vitamins, cells, antibodies, toxins, and pathogens (e.g., bacteria, viruses, fungus prion, or other disease-causing microorganism).

In some embodiments, the analyte is an inorganic compound. In some embodiments, the inorganic compound can be, but is not limited to gaseous inorganic compounds (e.g., carbon monoxide), chloroalkanes (e.g., carbon tetrachloride), or metal salts.

In some embodiments, the sensor is a biosensor for detecting an analyte that is a biomolecule. In some embodiments, the biomolecule is a carbohydrate, protein, nucleic acid, lipid, hormone, neurotransmitter, or vitamin. In an advantageous embodiment, the biomolecule is a hormone.

In some embodiments, the biosensor is configured to detect pharmaceutical analytes, which can include, but are not limited to controlled and addictive substances such as cannabinoids, amphetamines, opiates, and cocaine, as well as therapeutic agents such as antipsychotics, antibiotics, and analgesics.

The sample can be of any medium that contains the analyte of interest. In some embodiments, the sample can be a solid sample. In some embodiments, the sample can be a liquid sample. In some embodiments, the liquid sample can be an aqueous solution containing the analyte of interest. In some embodiments, the liquid sample can be an organic solution containing the analyte of interest.

In some embodiments, the sample is a biological sample. In some embodiments, the biological sample is a biofluid. In some embodiments, the biofluid can include, but is not limited to blood, serum, plasma, sweat, saliva, urine, stool, interstitial fluid, vaginal fluid, semen, bile, sputum, cerebrospinal fluid, or bone marrow aspirate.

In an advantageous embodiment, the sample is a sweat sample. In another advantageous embodiment, the sample is a saliva sample. In yet another advantageous embodiment, the sample is a blood sample.

In some embodiments, the sample is a tissue specimen. In some embodiments, the tissue specimen is a sample from a biopsy or a surgical resection. In some embodiments, the tissue sample is a tumor.

In some embodiments, the sample is a sample that has been further processed following collection for detection of the analyte utilizing the sensor described herein. In some embodiments, the further processing results in a solid or liquid sample containing the analyte of interest.

In some embodiments, the sample that has been further processed is a biological sample. In some embodiments, the sample that has been further processed can be a solution of a biofluid that contains the analyte of interest. In some embodiments, the biofluid can include, but is not limited to blood, serum, plasma, sweat, saliva, urine, stool, vaginal fluid, semen, bile, sputum, cerebrospinal fluid, or bone marrow aspirate.

In some embodiments, the sample is taken from an animal. In some embodiments, the animal is a mammal. In some embodiments, the mammal is a human. In an advantageous embodiment, the sample is taken from a human. In another advantageous embodiment, the sample is taken from an animal, which may include, but is not limited to a canine, feline, equine, bovine, ovine, swine, avian, or ursine. In yet another advantageous embodiment, the sample is taken from a domestic dog or a domestic cat. In another advantageous embodiment, the sample is taken from domestic livestock, which can include, for example horse, cattle, sheep, goat, donkey, chicken, rooster, pig, llama, or alpaca.

The sensor described herein exhibits enhanced sensitivity, enabling detection of analytes at very low concentrations whether the sample is analyzed in its native state or after processing (e.g., dilution, reconstitution, or transfer into an alternative medium). In certain embodiments, the limit of detection (LOD) can be as low as about 0.1 nM. In an advantageous embodiment, the dynamic detection range spans from approximately 1 nM to 1 mM in biofluids such as saliva, sweat, blood, interstitial fluid, and urine, thereby enabling detection of low-level, physiological-level, and elevated-level concentrations of the analyte of interest.

In some embodiments, the limits of detection can be about 0.1 nM, 0.2 nM, 0.3 nM, 0.4 nM 0.5 nM, 0.6 nM, 0.7 nM, 0.8 nM, 0.9 nM, 1 nM, 2 nM, 3 nM, 4 nM 5 nM, 6 nM, 7 nM, 8 nM, 9 nM, 10 nM, 15 nM, 20 nM, 25 nM, 30 nM, 35 nM, 40 nM, 45 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 5 μM, 10 μM, 15 μM, 20 μM, 25 μM, 30 μM, 35 μM, 40 μM, 45 μM, 50 μM, 75 μM, 100 μM, 200 μM, 300 μM, 400 μM, 500 μM, 600 μM, 700 μM, 800μM, 900μM, or about 1 mM.

In an advantageous embodiment, the analyte is cortisol and the detection range for cortisol as a biomarker in biofluids (e.g., blood, sweat, saliva, interstitial fluid) extends from about 0.5 nM to about 2000 nM, thereby covering cortisol deficiency, normal physiological levels, and elevated cortisol levels and permitting detection of the same (i.e. indications of cortisol deficiency, normal physiological levels of cortisol, and elevated cortisol levels).

In some embodiments, the sensor includes at least one working electrode formed on a substrate. The sensor may also include a counter electrode (or counter/reference electrode), and a reference electrode. The counter electrode and reference electrode may each be independently formed on the substrate or may be separate units.

In some embodiments, the working electrode is formed using conductive traces disposed on the substrate. In some embodiments, the counter electrode and reference electrode may also be formed using conductive traces disposed on the substrate. These conductive traces may be formed over a smooth surface of the substrate or within channels formed by, for example, embossing, indenting or otherwise creating a depression in the substrate.

Any technique may be used to fabricate the electrochemical cell as described herein and can include, but is not limited to screen-printing, inkjet printing, or photolithography. In an advantageous embodiment, the electrochemical cell is fabricated by screen-printing.

The working electrode can be constructed from materials including, but not limited to, carbon-based materials, which can include, for example, carbon ink, graphene, or carbon nanotubes, noble metals, which can include, for example, gold, platinum, doped metal oxides, which can include, for example, indium tin oxide, (ITO), or bismuth-based surfaces. The selection of the electrode material depends on the desired electrochemical properties and the specific functionalization method to be employed.

The working electrode can be of any size and the size utilized will depend on the overall application of the sensor. In some embodiments, the working electrode can have a diameter of about 0.1 mm, 0.2 mm, 0.3 mm, 0.4 mm, 0.5 mm, 0.6 mm, 0.7 mm, 0.8 mm, 0.9 mm, 1 mm, 1.5 mm, 2 mm, 2.5 mm, 3 mm, 3.5 mm, 4 mm, 4.5 mm, 5.5 mm, 6 mm, 6.5 mm, 7 mm, 7.5 mm, 8 mm, 8.5 mm, 9 mm, 9.5 mm, or about 10 mm. In an advantageous embodiment, the working electrode has a diameter of about 1 mm to about 3 mm to minimize the amount of sample needed to conduct the analysis.

The sensor as described herein permits detection and analysis of the target analyte via use of a small volume sample such that only a minimal amount of sample is required. The size of the sample will depend on the size of the working electrode, the number of working electrodes utilized in the analysis, as well as the concentration of analyte within the sample. In some embodiments, the sample size can be about 2 μl, 5 μl, 10 μl, 20 μl, 30 μl, 40 μl, 50 μl, 60 μl, 70 μl, 80 μl, 90 μl, 100 μl, 150 μl, 200 μl or about 250 μl.

The electrochemical cell similarly can be of any size that is appropriate for the desired application. In some embodiments, the length and width of the electrochemical cell can each independently be about 5 mm, 10 mm, 15 mm, 20 mm, 25 mm, 30 mm, 35 mm, 40 mm, 45 mm, 50 mm, 55 mm, or about 60 mm. In an advantageous embodiment, the electrochemical cell has a length of about 30 mm and a width of about 10 mm.

In some embodiments, a recognition layer can be formed proximate to or on the working electrode to facilitate the electrochemical detection of the analyte and the determination of its level in the sample.

In some embodiments, the recognition layer is configured to immobilize a receptor wherein the receptor has affinity for a specific analyte. In some such embodiments, the recognition layer can be made of any material that permits the immobilization of the receptor for binding to the analyte as described further herein.

In some embodiments, the recognition layer comprises a binding cavity that is complementary to the analyte. In other words, the binding cavity has a shape that is configured to accept the analyte in a “lock and key” type of model and binding interaction. In some such embodiments, the recognition layer can further comprise an electron transfer agent that permits the carrying electrons between the analyte and the working electrode as described further herein.

The substrate may be formed using a variety of non-conducting materials, including, for example, polymeric or plastic materials and ceramic materials. Suitable materials for a particular sensor may be determined, at least in part, based on the desired use of the sensor and properties of the materials.

In some embodiments, the substrate is flexible. For example, if the sensor is configured for implementation on a wearable device, then the sensor may be made flexible. A flexible substrate increases comfort and allows a wider range of activities. Suitable materials for a flexible substrate include, for example, non-conducting plastic or polymeric materials and other non-conducting, flexible, deformable materials. Examples of useful plastic or polymeric materials include thermoplastics such as polycarbonates, polyesters (e.g., Mylar™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate).

In some embodiments, the sensor can be made using a relatively rigid substrate to, for example, provide structural support against bending or breaking. Examples of rigid materials that may be used as the substrate include poorly conducting ceramics, such as aluminum oxide and silicon dioxide.

It will be appreciated that for many sensors and sensor applications, both rigid and flexible sensors will operate adequately. The flexibility of the sensor may also be controlled and varied along a continuum by changing, for example, the composition and/or thickness of the substrate.

In addition to considerations regarding flexibility, it is often desirable that the sensor should have a substrate that is non-toxic, especially if the sensor contacts the skin for the collection of the sample (e.g., sweat).

In some embodiments, the electrochemical cell of the sensor the conductive traces are connected to connection pads to interface with a measurement circuit. In some embodiments, the measurement circuit is a component of measurement electronics that permit measurement of a change in electrical property due to the binding of the analyte to the recognition layer. In an advantageous embodiment, the measurement electronics are a potentiostat.

In some embodiments, the measurement circuit applies an electric potential to the electrochemical cell and detects the change in the electrical property at the working electrode caused by the binding of the analyte to the recognition layer. In some embodiments, the measurement circuit measures the change in electrical property by a conformational change induced by the binding of the analyte to a receptor disposed on the recognition layer wherein said conformational change is correlative to a concentration of the analyte in the sample. In some embodiments, the measurement circuit measures the change in electrical property by the binding of the analyte to a binding cavity in the recognition layer in which said binding cavity is complementary to the analyte and the measured signal based on this binding of the analyte to the binding cavity is correlative to a concentration of the analyte in the sample.

The concentration of the analyte can be indicative of an underlying health condition or indication. For example, high or elevated levels of cortisol are indicative of higher levels of stress, which can lead to further health issues such as high blood pressure, high blood sugar, and weight gain. Thus, one objective of the invention as described herein is to provide a non-invasive, expedient, and accurate system to measure the concentration of such analytes that can be indicative of further underlying health issues.

As described herein, techniques that can be used to measure the change in electrical properties via the binding of the analyte to the recognition layer of the working electrode can include, but are not limited to electrochemical impedance spectroscopy (EIS), capacitance spectroscopy, field-effect transduction, cyclic voltammetry (CV), differential pulse voltammetry (DPV), square wave voltammetry (SWV), or amperometry.

In certain embodiments, determination of analyte concentration can be achieved by comparing the signals obtained from the sample of interest with signals generated from a series of standards of known analyte concentrations using the same analytical technique or instrumentation. A calibration curve is generated from the responses of the known standards, and the instrument response from the sample of interest is then correlated with the calibration curve, or its corresponding mathematical trendline or equation, to calculate the analyte concentration within the sample.

### Immobilized Nuclear Receptor

In some embodiments, the recognition layer can be configured to immobilize a receptor that has a specific affinity for a particular analyte.

In some embodiments, the receptor is a nuclear receptor. Nuclear receptors are molecules that regulate gene expression under the control of ligands such as hormones, steroids, and vitamins and are capable of detecting such ligands for regulation of gene expression. For example the estrogen receptor (ER) regulates the expression of estrogen dependent genes. The binding of a target ligand to a nuclear receptor triggers a change in conformation, thereby activating the nuclear receptor.

In some embodiments, the receptor can comprise a binding portion of a nuclear receptor wherein the binding portion is capable of both binding to the analyte and inducing a conformational change that leads to a change in electrical property to be detected by the measurement circuit. In an advantageous embodiment, the receptor is a full-length nuclear receptor.

In some embodiments, the nuclear receptor is selected from a glucocorticoid receptor (GR), estrogen receptor (ER), progesterone receptor (PR), androgen receptor (AR), mineralocorticoid receptor (MR), thyroid hormone receptor (TR), a peroxisome proliferator-activated receptor (PPAR), a liver X receptor (LXR), a farnesoid X receptor (FXR), a pregnane X receptor (PXR), or a vitamin D receptor (VDR). In some embodiments, the nuclear receptor is selected from the group consisting of a glucocorticoid receptor (GR), estrogen receptor (ER), progesterone receptor (PR), androgen receptor (AR), mineralocorticoid receptor (MR), thyroid hormone receptor (TR), a peroxisome proliferator-activated receptor (PPAR), a liver X receptor (LXR), a farnesoid X receptor (FXR), a pregnane X receptor (PXR), and a vitamin D receptor (VDR). In an advantageous embodiment, the nuclear receptor is a glucocorticoid receptor (GR).

Each of the nuclear receptors described herein has binding affinity for a specific analyte. For example, the Glucocorticoid Receptor (GR) has binding affinity for cortisol and can be used to detect cortisol, the Estrogen Receptor (ER) has binding affinity for estradiol and can be used for detecting estradiol, the Androgen Receptor (AR) has binding affinity for testosterone and can be used to detect testosterone. Accordingly in some embodiments, the analyte is selected from cortisol, estradiol, progesterone, testosterone, thyroid hormone, cholesterol, xenobiotics, vitamin D, dehydroepiandrosterone (DHEA), dopamine, serotonin, norepinephrine, epinephrine, uric acid, lactic acid, tryptophan, kynurenine, kynurenic acid, glutamate, gamma-aminobutyric acid (GABA), brain-derived neurotrophic factor (BDNF), C-reactive protein, IL-6, neuropeptide Y, oxytocin, melatonin, histamine, anandamide, α-amylase, cardiac troponin T, orexin A, or TNF-α. In some embodiments, the analyte is selected from the group consisting of cortisol, estradiol, progesterone, testosterone, thyroid hormone, cholesterol, xenobiotics, vitamin D, dehydroepiandrosterone (DHEA), dopamine, serotonin, norepinephrine, epinephrine, uric acid, lactic acid, tryptophan, kynurenine, kynurenic acid, glutamate, gamma-aminobutyric acid (GABA), brain-derived neurotrophic factor (BDNF), C-reactive protein, IL-6, neuropeptide Y, oxytocin, melatonin, histamine, anandamide, α-amylase, cardiac troponin T, orexin A, and TNF-α.

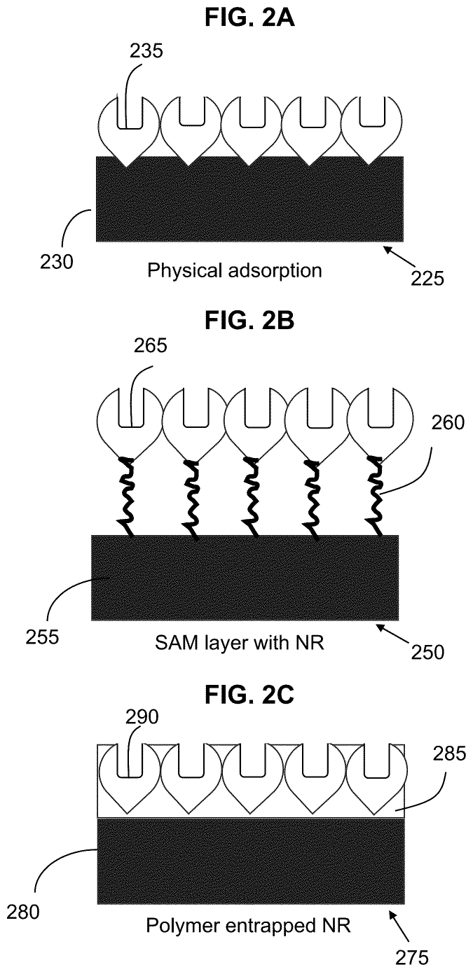

As previously discussed, in some embodiments, the receptor is immobilized onto the recognition layer. Any technique can be used to immobilize or otherwise anchor the receptor to the recognition layer and such examples are illustrated in FIGS. 2A-2C. In some embodiments, physical adsorption may be used to deposit the receptor onto the recognition layer. Physical adsorption involves the simple deposition of the receptor onto the recognition layer on the electrode surface as shown in FIG. 2A. In FIG. 2A, which depicts a sensor 225, the nuclear receptor 235 is deposited onto the substrate of the working electrode 230. In one embodiment, a solution containing the receptor may be cast onto the receptor layer and allowed to dry. In another such embodiment, the receptor may be deposited onto the recognition layer via sublimation from a premixed solution. If the particular working electrode includes more than one receptor or otherwise a combination of receptors, the evaporation temperature of each receptor utilized should be similar to ensure a more uniform distribution of receptor onto the recognition layer of the working electrode.

In some embodiments, covalent attachment may be used to immobilize the receptor onto the recognition layer. Covalent attachment involves utilizing a functionalized linker anchored to the substrate of the working electrode to provide a stable, oriented layer.

In some embodiments, a self-assembled monolayer (SAM) may be formed on an appropriate substrate wherein the SAM is configured to covalently bond to the receptor. For example, a SAM can be formed via the immobilization of a functionalized thiol onto a substrate in which the thiol includes a functional group that is capable of covalently bonding to the receptor. An example of a SAM-based system to immobilize the nuclear receptor is illustrated in FIG. 2B, which depicts a sensor 250 wherein a SAM 260 is anchored to the substrate of the working electrode 255 wherein the SAM has been functionalized to covalently bond the nuclear receptor 265.

Any substrate material that is suitable for forming the SAM can be used. In some embodiments, the substrate for forming the SAM can include, but is not limited to gold, silver, platinum, carbon. In an advantageous embodiment, the substrate on which the SAM is formed is gold.

The SAM can be of any thickness and depends on the compounds used to form the SAM and the requisite applications of the SAM. The SAM can have a thickness of about 0.1 nm, 0.2 nm, 0.3 nm, 0.4 m, 0.5 nm, 0.6 nm, 0.7 nm, 0.8 nm, 0.9 nm, 1 nm, 1.1 nm, 1.2 nm, 1.3 nm, 1.4 nm, 1.5 nm, 1.6 nm, 1.7 nm, 1.8 nm, 1.9 nm, 2 nm, 2.1 nm, 2.2 nm, 2.3 nm, 2.4 nm, 2.5 nm, 2.6 nm, 2.7 nm, 2.8 nm, 2.9 nm, 3 nm, 4 nm, 5 nm, 6 nm, 7 nm, 8 nm, 9 nm, 10 nm, 15 nm, 20 nm, 25 nm 50 nm, 75 nm, or about 100 nm. In an advantageous embodiment, the SAM has a thickness from about 1 nm to about 3 nm.

The thiol for forming the SAM can be, for example a hydroxyl-terminated thiol such as mercaptoundecanol (MUDOL), a carboxylate-terminated thiol such as 11-mercaptoundecanoic acid (MUDA), or combinations thereof. In the case of MUDA, covalent bonding to the receptor can be accomplished utilizing 1-ethyl-3-(3-dimethylaminopropyl) carbodiimide (EDC) and N-hydroxysuccinimide (NHS) to covalently bond the carboxylate groups of MUDA to amine groups of the receptor. In embodiments comprising a combination of MUDA and MUDOL, a molar ratio of MUDA to MUDOL can be about 1:1, 1.1:1, 1.2:1, 1.3:2, 1.4:1, 1.5:1, 2:1, 3:1, 4:1, 5:1, 6:1, 7:1, 8:1, 9:1, 10:1, 25:1, 50:1, 75:1, or about 100:1.

In some embodiments, diazonium electrografting can be used to covalently attach the receptor to the recognition layer on the electrode surface.

In some embodiments, the receptor can be entrapped in a polymer matrix. Biocompatible hydrogels such as chitosan, Nafion, or conductive polymers (e.g., polypyrrole) can be used to entrap the receptor on the recognition, thereby preserving its bioactivity. A sensor 275 having a polymer entrapped nuclear receptor is illustrated in FIG. 2C, in which a polymer 285, which contains the entrapped nuclear receptor 290, is formed or deposited onto the substrate of the working electrode 280. As further shown in FIG. 2C, the nuclear receptor 290 remains functional as the binding sites for the analyte remain open and accessible.

In some embodiments, affinity-based immobilization techniques can be utilized to immobilize the receptor on the recognition layer. Examples include His-Tag, Ni-NTA, or Biotin-Streptavidin binding, which allow for gentle, oriented immobilization and maintains the conformational integrity and function of the receptor.

In an advantageous embodiment, the receptor is immobilized on the recognition layer via covalent attachment. In another advantageous embodiment, the receptor is immobilized on a SAM wherein the SAM comprises functionalized thiols. In another advantageous embodiment, the thiols are MUDOL and MUDA. In some such embodiments, a molar ratio of MUDA to MUDOL is from about 1.1:1 to about 2:1. In some such embodiments, the receptor is covalently bonded to the carboxylate and hydroxyl groups of the MUDA and MUDOL, respectively.

In some embodiments, the receptor is a nuclear receptor that is immobilized on the recognition layer and the binding of the analyte to the nuclear receptor induces a conformational change that can be measured through application of the potential from the measurement circuit wherein the measured change in electrical property caused by the conformation change correlates to a concentration of the analyte in the sample.

### Molecularly Imprinted Polymer

In some embodiments, the recognition layer comprises a molecularly imprinted polymer (MIP) layer or a layer of nanoparticles disposed on the working electrode, the MIP comprising a binding cavity complementary to the analyte.

The MIP can be made of any polymer that is suitable for a particular application or for a particular sample type or medium. In some embodiments, the MIP can be made from phenol, o-aminophenol, o-phenylenediamine, aniline, scopoletin, pyrrole, 3,4-ethylenedioxythiophene (EDOT), dopamine, monomers comprising boronic acid groups, vinyl acetate, methyl methacrylate, methacrylic acid, styrene, acrylamide, methacrylamide, bis-acrylamide, acrylic acid, acrylamide, tert-butyl acrylate, N-(3-Aminopropyl)methacrylamide hydrochloride, or combinations or copolymers thereof. In an advantageous embodiment, electrodeposited MIP can be made from o-phenylenediamine.

In some embodiments, the MIP can further comprise an electron transfer agent to facilitate transfer of electrons from the analyte and the working electrode. Upon binding of the analyte to its complementary binding cavity in the MIP, the electron transfer agent can undergo a reaction to generate a signal at the working electrode (e.g., a Faradaic current), which can be measured by the measurement circuit.

In some embodiments the electron transfer agent is bound or otherwise immobilized on the working electrode or between or within the MIP disposed over the working electrode. The electron transfer agent may be immobilized on the working electrode using, for example, a polymeric or sol-gel immobilization technique. Alternatively, the electron transfer agent may be chemically (e.g., ionically, covalently, or coordinatively) bound to the working electrode, either directly or indirectly through another molecule, such as a polymer, that is in turn bound to the working electrode. In an advantageous embodiment, the electron transfer agent is embedded within the MIP layer.

The electron transfer agent may be organic, organometallic, or inorganic. Examples of organic redox species are quinones and species that in their oxidized state have quinoid structures, such as Nile blue and indophenol. Some quinones and partially oxidized quinhydrones react with functional groups of proteins such as the thiol groups of cysteine, the amine groups of lysine and arginine, and the phenolic groups of tyrosine which may render those redox species unsuitable for some of the sensors of the present invention because of the presence of the interfering proteins in an analyte-containing fluid. Usually substituted quinones and molecules with quinoid structure are less reactive with proteins and are preferred. A preferred tetrasubstituted quinone usually has carbon atoms in positions 1, 2, 3, and 4.

In general, electron transfer agents have structures or charges which prevent or substantially reduce the diffusional loss of the electron transfer agent during the period of time that the sample is being analyzed. The preferred electron transfer agents include a redox species embedded within the MIP that is disposed on the working electrode. The bond between the redox species and the MIP polymer may be covalent, coordinative, or ionic. Useful electron transfer agents and methods for producing them are described in U.S. Pat. Nos. 5,264,104; 5,356,786; 5,262,035; and 5,320,725, incorporated herein by reference. Although any organic or organometallic redox species can be used as an electron transfer agent, the preferred redox species is a transition metal compound or complex. The preferred transition metal compounds or complexes include osmium, ruthenium, iron, and cobalt compounds or complexes. The most preferred are osmium compounds and complexes. It will be recognized that many of the redox species described below may also be used as electron transfer agents in a carrier fluid or in a sensing layer of a sensor where leaching of the electron transfer agent is acceptable.

In some embodiments, the electron transfer agent is a redox-active catalyst that catalyzes a reaction of the analyte and generates a signal. In some embodiments the redox-active catalyst is an enzyme that catalyzes a reaction of the analyte. For example, a catalyst, such as a glucose oxidase, glucose dehydrogenase (e.g., pyrroloquinoline quinone glucose dehydrogenase (PQQ)), or oligosaccharide dehydrogenase, may be used when the analyte is glucose. A lactate oxidase or lactate dehydrogenase may be used when the analyte is lactate. Laccase may be used when the analyte is oxygen or when oxygen is generated or consumed in response to a reaction of the analyte.

In some embodiments, the redox-active catalyst is selected from platinum, gold, a metal oxide, a metal-organic framework (MOF), graphene, carbon nanotubes, carbon dots, metal nanoparticles, single metal atoms, porphyrins, phthalocyanines, and combinations thereof. In an advantageous embodiment, the redox-active catalyst is copper phthalocyanine.

The MIP can have any thickness as long as it is able to provide the binding cavities complementary to the analyte and, if present, accommodate the presence of the electron transfer agent. In some embodiments, the MIP can have a thickness of about 10 nm, 20 nm, 30 nm, 40 nm, 50 nm, 60 nm, 70 nm, 80 nm, 90 nm, 100 nm, 1110 nm, 20 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 325 nm, 350 nm, 375 nm, 400 nm, 425 nm, 450 nm, 475 nm, 500 nm, 550 nm, 600 nm, 700 nm, 800 nm, 900 nm, or about 1000 nm. In an advantageous embodiment, the thickness of the MIP is from about 100 nm to about 300 nm.

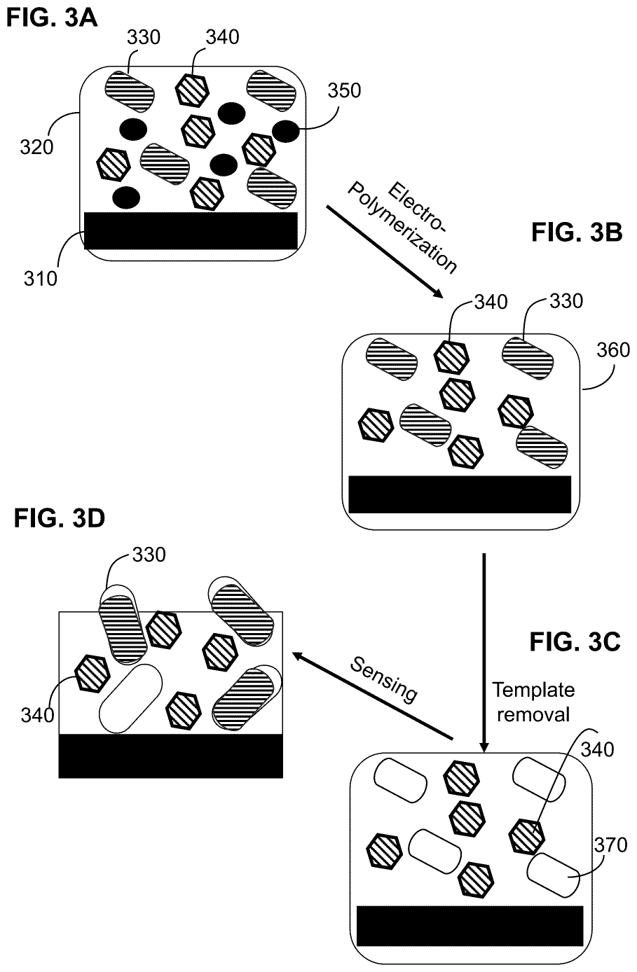

The MIP can be prepared by any method known in the art that is suitable for embedding the template molecules (e.g., the target biomarker or analyte) along with the electron transfer agent or redox-active catalyst to catalyze a reaction with the bonded analyte and generate a signal that can be measured. In some embodiments, as illustrated in FIGS. 3A-3C, a process for creating an electrodeposited MIP can include the steps of:
preparing a solution 320 containing one or more functional monomers 350, the target biomarker acting as a template molecule 330, and a redox-active catalyst 340;
electro-polymerizing said solution directly onto the surface of the electrode to form a thin polymer film 360 as shown in FIG. 3B, with the template molecules 330 and redox-active catalyst 340 embedded within; and
removing the template molecules 330, thereby forming biomarker-specific binding cavities 370 that are complementary in size, shape, and functionality to the target analyte, wherein the catalyst 340 remains integrated within the polymer matrix as depicted in FIG. 3C.

As shown in FIG. 3D, in operation, sensing occurs via the binding of the analyte or target biomarker 330 to the binding cavity 370.

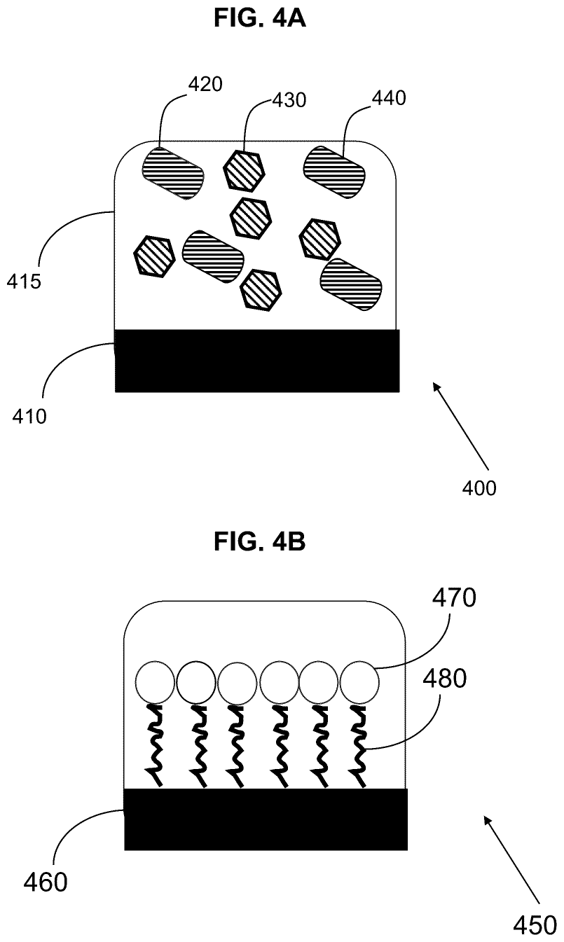

FIG. 4A shows a working electrode 400 having an electrodeposited MIP 415 that has been deposited on the substrate 410 of the working electrode. The electrodeposited MIP 415 contains the binding cavity 440 for the analyte or biomarker 420 wherein the MIP contains embedded redox-active catalyst 430 to catalyze an oxidation or reduction reaction with the analyte, thereby generating a signal that can be measured based on the binding interaction of the analyte with the binding cavity.

In some embodiments, as illustrated in FIG. 4B, the MIP can be prepared in solution as particles 470 featuring cavities configured to bind to the analyte wherein the MIP particles can be subsequently immobilized on the substrate 460 of the electrode via a linker 480 using any applicable technique including those as described herein. As depicted in the working electrode 450 of FIG. 4B, the linkers can be part of a SAM. The MIP particles 470 can further contain embedded redox-active catalyst and catalyze an oxidation or reduction reaction upon binding to the analyte and generate a signal that can be measured as described herein.

### Sensing Mechanisms

As discussed above, the measured binding interaction between the analyte and the recognition layer will depend on the binding and recognition system that is utilized in the sensor receptor (e.g., a receptor such as a nuclear receptor or via a binding cavity in a MIP).

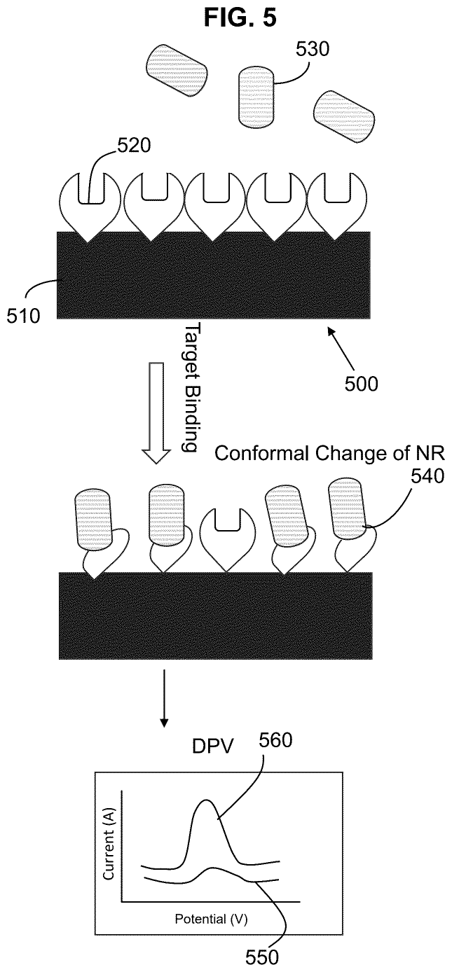

In some embodiments, the analyte binds to the recognition layer via a receptor such as a nuclear receptor. FIG. 5 depicts the binding of the biomarker or analyte 530 to the nuclear receptor 520, wherein the nuclear receptor is immobilized on the substrate 510 of the working electrode 500. The binding of a target biomarker (e.g., the analyte) 530 to the ligand-binding domain of the immobilized nuclear receptor 520 induces a significant conformational change 540 in the protein. This conformational change 540 alters the electrical double layer and charge distribution at the electrode interface and/or may detach a receptor-target complex from the electrode substrate, thereby directly modulating electrochemical properties such as impedance or capacitance. This label-free transduction can be measured sensitively using any applicable technique, which can include, for example, electrochemical impedance spectroscopy (EIS) or capacitance spectroscopy. Alternatively, the change in conformation may be measured in the presence of a redox probe solution (e.g., ferricyanide/ferrocyanide) using techniques such as Differential Pulse Voltammetry (DPV), wherein a shift in the voltammetric signal before 550 and after 560 sample introduction correlates to analyte binding.

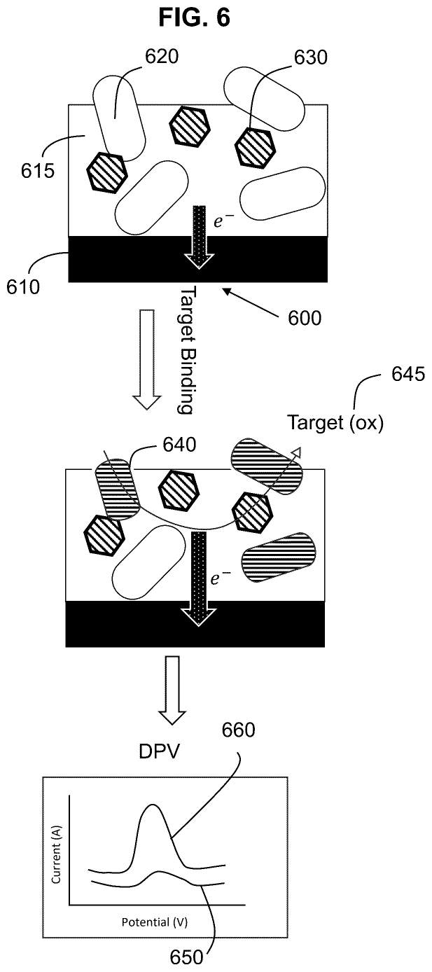

In some embodiments, as depicted in FIG. 6, a MIP 615 is deposited on the surface of the substrate 610 of the working electrode 600. In some such embodiments, the analyte binds to a complementary binding cavity on a MIP. In some embodiments, upon binding of a target biomarker (e.g., the analyte) 620 into its complementary binding cavity 640 of the MIP 615 containing an embedded or otherwise integrated redox-catalyst 630, the integrated redox-active catalyst (e.g., copper phthalocyanine) facilitates the selective oxidation or reduction 645 of the analyte. This catalytic reaction 645 generates a signal (e.g., a Faradaic current) that can be measured directly, without the need for external redox mediators. Techniques such as Differential Pulse Voltammetry (DPV) can be used to measure the signal, wherein a shift in the voltammetric signal before 650 and after 660 sample introduction correlates to analyte binding.

### Assays

The sensor as described herein can be supported in multiple assay formats. In some embodiments, the sensor as described herein can be utilize in a label-free direct binding assay. In the label-free assay, the sample is applied directly to the functionalized working electrode. For working electrodes that have been functionalized with a nuclear receptor, the conformational change is detected directly via EIS or capacitance spectroscopy.

In some embodiments, the sensor as described herein can be utilized in a redox-mediated assay. In the redox-mediated assay, a redox probe in solution (e.g., ferricyanide/ferrocyanide) or embedded within the working electrode is used. The binding event (e.g., the conformational change induced from the analyte binding to the nuclear receptor or MIP cavity occupancy) alters the electron transfer efficiency of the probe, which is measured sensitively using DPV or Square Wave Voltammetry (SWV).

In some embodiments, the sensor as described herein can be utilized in a competitive binding assay. In a competitive binding assay, a captured version of the analyte or an analog thereof is immobilized on the working electrode. The sample is pre-mixed with a known quantity of free nuclear receptor. The free analyte in the sample and the immobilized analyte compete for the limited binding sites on the receptor. The amount of receptor subsequently bound to the electrode surface, detected via its intrinsic conformational change, is inversely proportional to the concentration of the target analyte in the sample.

### Multiplex Systems

In some embodiments, the sensor can comprise a plurality of working electrodes,
wherein each of the plurality of working electrodes includes a recognition layer that is configured to bind to an analyte;
wherein each recognition layer is different from each other; and
wherein each recognition layer binds to a different analyte than the analyte of the other recognition layers.

What is meant by the recognition layers being different from each other is that each of the recognition layers has an affinity for a different analyte than that of the other recognition layers. The structural, composition of each of the recognition layers can be same or different from each other as long as the target analyte for each recognition layer is different.

In other words, in some embodiments, each recognition layer of each of the plurality of working electrodes can independently be, for example a SAM having an immobilized nuclear receptor or a MIP having a binding cavity that is complementary for a specific analyte as long as each recognition layer, regardless of comprising the SAM having the immobilized nuclear receptor or the MIP configured to complementarily bind to an analyte binds to a different analyte. In some embodiments, each of the plurality of working electrodes comprises a SAM having an immobilized nuclear receptor for binding a specific analyte. In some embodiments, each of the plurality of working electrodes comprises a MIP having a binding cavity that is complementary for each analyte and a redox-active catalyst. In some embodiments, at least one working electrode of the plurality of working electrodes comprises the SAM and at least one working electrode of the plurality of working electrodes comprises the MIP.

The ability to include and incorporate multiple working electrodes in an array in which each of the plurality of working electrodes can be functionalized with a different nuclear receptor or MIP for a different analyte permits detection and measurement of multiple analytes from a single, small volume sample. In some embodiments, a single sensor strip or cartridge can incorporate an array of working electrodes, each functionalized with a different nuclear receptor or MIP specific to a different biomarker. In some embodiments, a multiplexer and potentiostat system can be configured to sequentially or simultaneously apply potentials and measure electrochemical signals from each working electrode of the array of working electrodes to enable the parallel detection of a panel of biomarkers from a single, small-volume sample.

In some embodiments, the sensor is for point-of-care use and can comprise a sensor cartridge such as a screen-printed electrode strip, and a reusable reader/hub containing a low-power potentiostat, with wired or wireless communication ability for transmitting data to a mobile device or cloud platform to facilitate real-time health monitoring. In some embodiments, the sensor cartridge comprising the working electrode is disposable and single use. In some embodiments, the sensor can be integrated into a device, which can include, for example, a handheld reader, a wearable sweat-sensing patch with integrated microfluidics, or mouthguards for saliva monitoring.

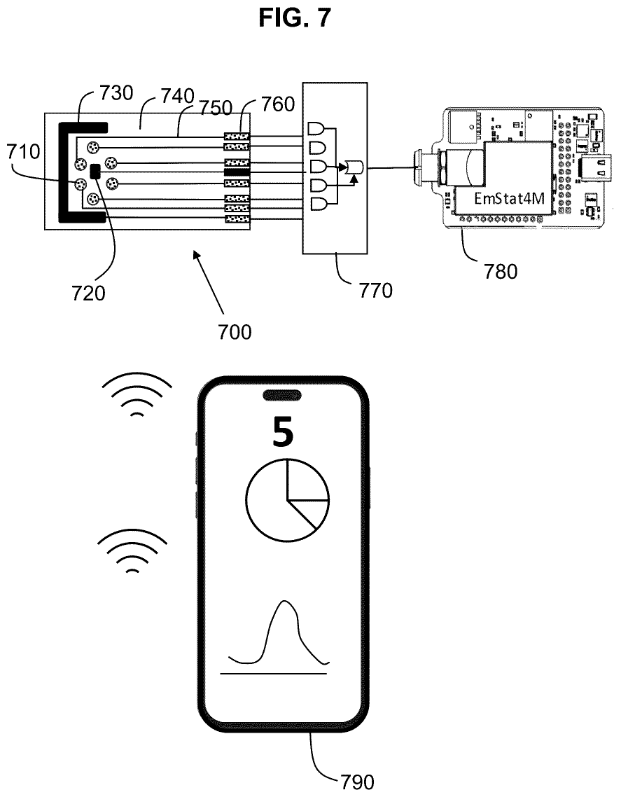

FIG. 7 provides an illustration of a multiplexed sensor. The multiplexed sensor contains a plurality of working electrodes disposed on a sensor array that can be coupled with a potentiostat for application of a potential to measure the electrochemical signals at each working electrode of the plurality of working electrodes. These signals can be transmitted wirelessly to a mobile device for further monitoring and processing of the results.

According to another aspect, a method for detecting an analyte in a sample is provided, wherein the method comprises the steps of:
contacting the sample with a sensor comprising an electrochemical cell;
the electrochemical cell comprising a working electrode, the working electrode comprising a nuclear receptor or a molecularly imprinted polymer (MIP) comprising a binding cavity that is complementary to the analyte;
binding the analyte to the nuclear receptor or to the binding cavity of the MIP;
applying a potential to the working electrode;
measuring, via a measurement circuit that is operably coupled to the electrochemical cell, a change in an electrical property at the working electrode, wherein the change in the electrical property is caused by a conformational change of the nuclear receptor induced by binding to the analyte or by catalytic redox activity induced by binding of the analyte to the binding cavity of the MIP; and
correlating the measured change to a concentration of the analyte in the sample.

In some embodiments of the method, the working electrode comprises the MIP, wherein the MIP comprises an integrated catalyst, and binding of the analyte to the binding cavity promotes selective oxidation or reduction of the analyte without requiring an external redox mediator

In some embodiments, the method comprises measuring the change in electrical property via electrochemical impedance spectroscopy (EIS), capacitance spectroscopy, or differential pulse voltammetry (DPV) as described herein.

According to another aspect, a competitive binding sensor for detecting an analyte in a liquid sample is provided, wherein the competitive binding sensor comprises: a working electrode functionalized with a captured version of the analyte or an analog thereof; a reservoir containing a known quantity of a nuclear receptor protein; a fluidic network configured to mix the liquid sample with the nuclear receptor to form a mixture and deliver the mixture to the working electrode; and
a measurement circuit configured to:
(i) apply a potential to the working electrode, and
(ii) measure an electrochemical signal,
wherein the magnitude of the electrochemical signal is inversely proportional to a concentration of the analyte in the liquid sample due to competitive binding between the analyte in the sample and the captured analyte for the nuclear receptor.

According to yet another aspect, a multiplexed biosensing system is provided, wherein the system comprises: a cartridge interface for receiving a biological sample; a fluidic network configured to distribute the sample to a plurality of electrochemical cells; wherein each electrochemical cell comprises a working electrode functionalized with a different recognition element specific to a different analyte, and wherein at least one recognition element comprises a full-length nuclear receptor and at least one recognition element comprises a molecularly imprinted polymer with an integrated catalyst; and a potentiostat unit configured to independently apply a potential to and measure an electrochemical signal from each of the plurality of working electrodes to simultaneously determine concentrations of the plurality of analytes.

The multiplex system containing different working electrodes having binding affinity and specificity for different analytes enables the parallel and simultaneous analysis of different analytes that may be present in the same sample (e.g., saliva, sweat). In some embodiments, the multiplex system can be utilized to measure different analytes from different samples via contacting each of the plurality of electrochemical cells with a different sample. Thus, for example, in some embodiments, a first working electrode can be utilized to detect an analyte found in sweat, a second working electrode can be utilized to detect an analyte found in saliva, and a third analyte can be utilized to detect an analyte found in blood. The ability to utilize the same system to obtain measurements as to various biomarkers can provide a more comprehensive screening on the health and well-being of a subject without having to utilize multiple systems and multiple techniques to achieve a similar result.

Depending on the analyte to be measured, the preferred mode of analyte binding and detection, and the overall limits of detection for the analyte, the size of each of the working electrodes of the multiplex system can be the same or different from each other.

The modes of sensing the analytes of interest can also be varied in the multiplex system to be tailored to the best modes for capturing and detecting a specific analyte. Thus, in some embodiments of the multiplex system, at least one working electrode contains a nuclear receptor for binding to the analyte. In some embodiments of the multiplex system, at least one working electrode contains a MIP having a binding cavity complementary to the analyte for binding to the analyte.

An example of a multiplex system is illustrated in FIG. 7, which shows a sensor array 700 containing a plurality of working electrodes 710 that are disposed (e.g., screen-printed) on a non-conductive surface 740 of the sensor array 700. The sensor array 700 also contains a reference electrode 720 and a counter electrode 730. Each of the electrodes is connectively coupled to a multiplexer 770 via conductive leads 750 and conductive pads 760. After contacting with a sample or samples containing the analytes of interest, the multiplexer 770 can be connectively coupled or inserted into a means for applying a potential across the sensor array 700, such as potentiostat 780, to measure the change in electrical properties arising from the binding of the analyte to the respective recognition layers of the working electrodes. The results measured from the potentiostat 780 can be transmitted (e.g., wirelessly over WiFi, or via a connective means such as a cable connected the measurement means) to a mobile device 790 or other means of visually displaying the results, which may be utilized to further monitor the health of the subject.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1—Cortisol Detection in Saliva

An exemplary embodiment for detecting cortisol in saliva is described. The experiment utilizes a screen-printed carbon electrode having at least two working electrodes. On a first working electrode, a Glucocorticoid Receptor (GR) is immobilized via physical adsorption. More in particular, a 10 μL solution of GR is dropcasted onto porous gold electrode for overnight passivation. After passivation, the GR solution is removed and the working electrode is dried. On a second working electrode, an electrodeposited MIP film having a binding cavity for cortisol is deposited onto the substrate of the second working electrode wherein the MIP further includes an integrated copper phthalocyanine catalyst that is immobilized within the MIP during the electrodeposition process.

The sample is human saliva. Approximately 50 μL of the collected saliva sample is diluted with phosphate-buffered saline (PBS) and contacted with the working electrode, where it is allowed to equilibrate for about 10 minutes.

After 10 minutes, detection of cortisol from the saliva sample at the first and second working electrodes is accomplished using electrochemical impedance spectroscopy (EIS) in saliva, which shows a linear response to cortisol across a physiologically relevant range from 1 nM to 100 nM. The sensor exhibits high selectivity against common interferents such as cortisone, demonstrating the effectiveness of the platform.

The invention is further described by the following numbered paragraphs:

1. A sensor for detection of an analyte in a sample, wherein the sensor comprises:
a substrate;
an electrochemical cell disposed on the substrate, wherein the electrochemical cell comprises:
   a working electrode comprising:
      a recognition layer disposed on a surface of the working electrode capable of binding to the analyte;
   a reference electrode; and
   a counter electrode;
   wherein binding of the analyte to the recognition layer comprises:
      binding of the analyte to a nuclear receptor immobilized on the recognition layer thereby inducing a conformation change; or
      binding of the analyte to a binding cavity complementary to the analyte;
and
a measurement circuit operably coupled to the electrochemical cell wherein the measurement circuit is configured to:
   i) apply a potential to the working electrode; and
   ii) detect a change in an electrical property at the working electrode caused by the binding of the analyte to the recognition layer, wherein said binding is correlative to a concentration of the analyte in the sample.

2. The sensor of paragraph 1, wherein the sensor comprises the nuclear receptor, wherein the nuclear receptor is selected from a glucocorticoid receptor (GR), estrogen receptor (ER), progesterone receptor (PR), androgen receptor (AR), mineralocorticoid receptor (MR), thyroid hormone receptor (TR), a peroxisome proliferator-activated receptor (PPAR), a liver X receptor (LXR), a farnesoid X receptor (FXR), a pregnane X receptor (PXR), or a vitamin D receptor (VDR).

3. The sensor of paragraph 1, wherein the analyte is selected from cortisol, estradiol, progesterone, testosterone, thyroid hormone, cholesterol, xenobiotics, or vitamin D.

4. The sensor of paragraph 1, wherein the sample is selected from saliva, sweat, urine, blood, serum, plasma, or interstitial fluid.

5. The sensor of paragraph 1, wherein the measurement circuit is configured to detect the change in the electrical property via electrochemical impedance spectroscopy (EIS), capacitance spectroscopy, field-effect transduction, cyclic voltammetry (CV), differential pulse voltammetry (DPV), square wave voltammetry (SWV), or amperometry.

6. The sensor of paragraph 1, comprising a plurality of working electrodes, wherein each of the plurality of working electrodes is functionalized with a receptor;
wherein each receptor is different from each other; and
wherein each receptor binds to a different analyte than the analyte of the other receptors.

7. The sensor of paragraph 6, wherein the receptor of each of the plurality of working electrodes comprises the nuclear receptor.

8. The sensor of paragraph 2, wherein:
the analyte comprises cortisol;
the nuclear receptor comprises a glucocorticoid receptor (GR);
wherein the GR is immobilized to the recognition layer.

9. The sensor of paragraph 8, wherein the GR is immobilized to the recognition layer via physical adsorption.

10. The sensor of paragraph 8, wherein the recognition layer comprises a self-assembled monolayer (SAM), wherein the SAM is functionalized for the covalent bonding of the GR to the SAM.

11. The sensor of paragraph 10, wherein the SAM is functionalized with a terminal reactive group selected from hydroxyl, carboxyl, amine, or thiol, and wherein the GR is covalently bonded to said terminal reactive group directly or through a bifunctional crosslinking agent.

12. The sensor of paragraph 11, wherein the SAM comprises mercaptoundecanol (MUDOL), 11-mercaptoundecanoic acid (MUDA), or combinations thereof.

13. The sensor of paragraph 12, comprising MUDA and MUDOL at a molar ratio of MUDA to MUDOL from about 1:1 to 2:1.

14. The sensor of paragraph 1, wherein the sensor is integrated into a wearable device selected from a transdermal patch, a wristband, a tooth-mounted sensor, or a smart textile.

15. The sensor of paragraph 1, further comprising a wireless communication module configured to transmit data corresponding to the analyte concentration to a remote computing device.

16. The sensor of paragraph 1, wherein the recognition layer comprises a molecularly imprinted polymer (MIP) layer or a layer of nanoparticles disposed on the working electrode, the MIP comprising a binding cavity complementary to the analyte.

17. The sensor of paragraph 16, wherein the MIP further comprises a redox-active catalyst selected from platinum, gold, a metal oxide, a metal-organic framework, graphene, carbon nanotubes, carbon dots, metal nanoparticles, single metal atoms, porphyrins, phthalocyanines, or combinations thereof.

18. The sensor of paragraph 17, wherein the catalyst interacts with the analyte captured in the binding cavity to generate a selective electrochemical signal without requiring an external redox mediator.

19. The sensor of paragraph 16, wherein the MIP is fabricated by a method selected from emulsion polymerization, precipitation polymerization, sol-gel polymerization, or electrodeposition.

20. The sensor of paragraph 16, wherein the sensor is configured to operate in a dual-receptor mode in which signals from both the nuclear receptor and the MIP are measured independently or synergistically.

21. The sensor of paragraph 17, wherein the analyte is selected from cortisol, estradiol, progesterone, testosterone, thyroid hormone, vitamin D, dehydroepiandrosterone (DHEA), dopamine, serotonin, norepinephrine, epinephrine, uric acid, lactic acid, tryptophan, kynurenine, kynurenic acid, glutamate, gamma-aminobutyric acid (GABA), brain-derived neurotrophic factor (BDNF), C-reactive protein, IL-6, neuropeptide Y, oxytocin, melatonin, histamine, anandamide, α-amylase, cardiac troponin T, orexin A, or TNF-α.

22. A method for detecting an analyte in a sample, the method comprising the steps of:
contacting the sample with a sensor comprising an electrochemical cell;
   the electrochemical cell comprising a working electrode, the working electrode comprising a nuclear receptor or a molecularly imprinted polymer (MIP) comprising a binding cavity that is complementary to the analyte;
binding the analyte to the nuclear receptor or to the binding cavity of the MIP;
applying a potential to the working electrode;
measuring, via a measurement circuit that is operably coupled to the electrochemical cell, a change in an electrical property at the working electrode, wherein the change in the electrical property is caused by a conformational change of the nuclear receptor induced by binding to the analyte or by catalytic redox activity induced by binding of the analyte to the binding cavity of the MIP; and
correlating the measured change to a concentration of the analyte in the sample.

23. The method of paragraph 22, wherein the MIP comprises an integrated catalyst, and binding of the analyte to the binding cavity promotes selective oxidation or reduction of the analyte without requiring a redox mediator.

24. A competitive binding sensor for detecting an analyte in a liquid sample, the sensor comprising: a working electrode functionalized with a captured version of the analyte or an analog thereof; a reservoir containing a known quantity of a nuclear receptor protein; a fluidic network configured to mix the liquid sample with the nuclear receptor to form a mixture and deliver the mixture to the working electrode; and
a measurement circuit configured to:
(i) apply a potential to the working electrode, and
(ii) measure an electrochemical signal,
wherein the magnitude of the electrochemical signal is inversely proportional to a concentration of the analyte in the liquid sample due to competitive binding between the analyte in the sample and the captured analyte for the nuclear receptor.

25. A multiplexed sensing system comprising: a cartridge interface for receiving a biological sample; a fluidic network configured to distribute the sample to a plurality of electrochemical cells; wherein each electrochemical cell comprises a working electrode functionalized with a different recognition element specific to a different analyte; and a potentiostat unit configured to independently apply a potential to and measure an electrochemical signal from each of the plurality of working electrodes to simultaneously determine concentrations of the plurality of analytes.

26. The system of paragraph 25, wherein at least one recognition element comprises a full-length nuclear receptor.

27. The system of paragraph 25, wherein at least one recognition element comprises a molecularly imprinted polymer with an integrated catalyst.

28. The system of paragraph 26, wherein the full-length nuclear receptor is a glucocorticoid receptor (GR) and the analyte comprises cortisol.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A sensor for detection of an analyte in a sample, wherein the sensor comprises:
a substrate;
an electrochemical cell disposed on the substrate, wherein the electrochemical cell comprises:
a working electrode comprising:
a recognition layer disposed on a surface of the working electrode capable of binding to the analyte;
a reference electrode; and
a counter electrode;
wherein binding of the analyte to the recognition layer comprises:
binding of the analyte to a nuclear receptor immobilized on the recognition layer thereby inducing a conformation change; or
binding of the analyte to a binding cavity complementary to the analyte; and
a measurement circuit operably coupled to the electrochemical cell wherein the measurement circuit is configured to:
i) apply a potential to the working electrode; and
ii) detect a change in an electrical property at the working electrode caused by the binding of the analyte to the recognition layer, wherein said binding is correlative to a concentration of the analyte in the sample.

2. The sensor of claim 1, wherein the sensor comprises the nuclear receptor, wherein the nuclear receptor is selected from a glucocorticoid receptor (GR), estrogen receptor (ER), progesterone receptor (PR), androgen receptor (AR), mineralocorticoid receptor (MR), thyroid hormone receptor (TR), a peroxisome proliferator-activated receptor (PPAR), a liver X receptor (LXR), a farnesoid X receptor (FXR), a pregnane X receptor (PXR), or a vitamin D receptor (VDR).

3. The sensor of claim 1, wherein the analyte is selected from cortisol, estradiol, progesterone, testosterone, thyroid hormone, cholesterol, xenobiotics, or vitamin D.

4. The sensor of claim 1, wherein the sample is selected from saliva, sweat, urine, blood, serum, plasma, or interstitial fluid.

5. The sensor of claim 1, wherein the measurement circuit is configured to detect the change in the electrical property via electrochemical impedance spectroscopy (EIS), capacitance spectroscopy, field-effect transduction, cyclic voltammetry (CV), differential pulse voltammetry (DPV), square wave voltammetry (SWV), or amperometry.

6. The sensor of claim 1, comprising a plurality of working electrodes, wherein each of the plurality of working electrodes is functionalized with a receptor;
wherein each receptor is different from each other; and
wherein each receptor binds to a different analyte than the analyte of the other receptors.

7. The sensor of claim 6, wherein the receptor of each of the plurality of working electrodes comprises the nuclear receptor.

8. The sensor of claim 2, wherein:
the analyte comprises cortisol;
the nuclear receptor comprises a glucocorticoid receptor (GR);
wherein the GR is immobilized to the recognition layer.

9. The sensor of claim 8, wherein the GR is immobilized to the recognition layer via physical adsorption.

10. The sensor of claim 8, wherein the recognition layer comprises a self-assembled monolayer (SAM), wherein the SAM is functionalized for the covalent bonding of the GR to the SAM.

11. The sensor of claim 10, wherein the SAM is functionalized with a terminal reactive group selected from hydroxyl, carboxyl, amine, or thiol, and wherein the GR is covalently bonded to said terminal reactive group directly or through a bifunctional crosslinking agent.

12. The sensor of claim 11, wherein the SAM comprises mercaptoundecanol (MUDOL), 11-mercaptoundecanoic acid (MUDA), or combinations thereof.

13. The sensor of claim 12, comprising MUDA and MUDOL at a molar ratio of MUDA to MUDOL from about 1:1 to 2:1.

14. The sensor of claim 1, wherein the sensor is integrated into a wearable device selected from a transdermal patch, a wristband, a tooth-mounted sensor, or a smart textile.

15. The sensor of claim 1, further comprising a wireless communication module configured to transmit data corresponding to the analyte concentration to a remote computing device.

16. The sensor of claim 1, wherein the recognition layer comprises a molecularly imprinted polymer (MIP) layer or a layer of nanoparticles disposed on the working electrode, the MIP comprising a binding cavity complementary to the analyte.

17. The sensor of claim 16, wherein the MIP further comprises a redox-active catalyst selected from platinum, gold, a metal oxide, a metal-organic framework, graphene, carbon nanotubes, carbon dots, metal nanoparticles, single metal atoms, porphyrins, phthalocyanines, or combinations thereof.

18. The sensor of claim 17, wherein the catalyst interacts with the analyte captured in the binding cavity to generate a selective electrochemical signal without requiring an external redox mediator.

19. The sensor of claim 16, wherein the MIP is fabricated by a method selected from emulsion polymerization, precipitation polymerization, sol-gel polymerization, or electrodeposition.

20. The sensor of claim 16, wherein the sensor is configured to operate in a dual-receptor mode in which signals from both the nuclear receptor and the MIP are measured independently or synergistically.

21. The sensor of claim 17, wherein the analyte is selected from cortisol, estradiol, progesterone, testosterone, thyroid hormone, vitamin D, dehydroepiandrosterone (DHEA), dopamine, serotonin, norepinephrine, epinephrine, uric acid, lactic acid, tryptophan, kynurenine, kynurenic acid, glutamate, gamma-aminobutyric acid (GABA), brain-derived neurotrophic factor (BDNF), C-reactive protein, IL-6, neuropeptide Y, oxytocin, melatonin, histamine, anandamide, α-amylase, cardiac troponin T, orexin A, or TNF-α.

22.-28. (canceled)
